(19) United States
(12) Patent Application Publication
(10) Pub. No.: US 2007/0219413 A1
(43) Pub. Date: Sep. 20, 2007

(54) Capsulated endoscope with memory storage device

(76) Inventor: Feng Chuan Lin, Bade City (TW)

Correspondence Address: TROXELL LAW OFFICE PLLC, SUITE 1404, 5205 LEESBURG PIKE, FALLS CHURCH, VA, 22041, US

(21) Appl. No.: 11/378,331

(22) Filed: Mar. 20, 2006

(51) Int. Cl. A61B 1/06; A61B 1/04

(52) U.S. Cl. 600/160; 600/109; 600/179; 600/118

(57) ABSTRACT

A capsulated endoscope includes a capsular main body of 11×26 mm in size, which is internally provided with a controller connected to a computer host interface, a image-sensor chip connected to the controller, a plurality of LEDs, a memory module, and a battery. The capsular main body is acid and alkali resisting, and includes a front optically transparent dome to serve as a viewing window. When the capsular main body is swallowed for conducting an examination, the image-sensor chip starts taking pictures in the digestive tract. When the examination is completed, the capsular main body is discharged via the anus. The discharged capsular main body is washed and disinfected and then cut open for connecting the controller to an external computer host via the computer host interface, so that the computer host is allowed to access image data stored on the memory module for displaying on a monitor.

### FIELD OF THE INVENTION

The present invention relates to an image capturing device, and more particularly, to a capsulated endoscope with memory storage device for taking pictures in human digestive system.

### BACKGROUND OF THE INVENTION

The examination of digestive tract has been conducted by many senior doctors since year 1795. In the early stage, the examination instrument for this purpose was relatively rough and inconvenient for use, and could normally be used to examine only the beginning and ending parts of the digestive tract. Later, an endoscope was invented in an attempt to facilitate and improve the examination of digestive tract. To examine a patient's digestive tract using the conventional endoscope, a duct is inserted into an organ to be examined. The organ is internally illuminated by cold light transmitted from a cold light source, so that a image-sensor chip, such as a charge-coupled device (CCD) that is similar to digital video camera (DV) or V8 video camera, provided at a distal end of the duct could clearly send image signals back to a machine, at where a built-in computer converts the image signals into pictures. However, there is still a considerably long section of the small intestine that could not be easily examined with the

conventional endoscope. This is because the conventional endoscope is difficult to handle, and the patient undergoing the examination would suffer greater

pain, if the duct therefor is too long.

With the highly advanced scientific technologies, a wireless biotelemetry is developed to take the place of the conventional endoscope that uses wired signal transmission, has a considerably big volume, and is inconvenient for use. Thanks to the invention of the energy-saving and miniaturized image-sensor chip, as well as the wireless transmitter, a compact and light-weight capsular endoscope has been developed in recent years. In the existing capsular endoscope, the image-sensor chip and cold light source for taking pictures are dimensionally reduced, enabling the endoscope to have a small size of 11×26 mm, which is close to a general capsule. In addition to the image-sensor chip, there are also cells, light-emitting diodes (LEDs), controller chip, and radio transmitter contained in the capsular endoscope, enabling the latter to take two pictures per second. In each examination requiring about 8 hours, total fifty thousand sheets of pictures are taken.

To conduct the examination of digestive tract using the capsular endoscope, the whole capsular endoscope is swallowed to move forward with the peristalsis of intestine. While the capsular endoscope moves forward, it flashes twice per second for the image-sensor chip to take pictures inside the organs at the same time. The image signals of the pictures taken by the image-sensor chip are then sent to an outer side of the patient's body by radio transmission. Nine pieces of radio receiving antennas are mounted at the abdomen of the patient undergoing the examination, and the received signals are stored on a portable receiver. The capsular endoscope faithfully records every picture it takes in the patient's body, from the esophagus to the small intestine or even the large intestine, until the cells therein are exhausted. The capsular endoscope is eventually discharged from the anus along with excrements to complete the whole examination. Generally speaking, the examination of digestive tract using the capsular endoscope can be conducted without causing too much inconvenience to the patient.

However, the capsular endoscope utilizing wireless data transmission and the peripherals thereof are extremely expensive; and the big-volume antennas and signal receiver mounted on the patient's body for receiving and storing the image data must be installed only by a trained professional through complicate procedures. These big-volume antennas and signal receiver would inevitably restrict the patient from moving conveniently. All these factors prevent the capsular endoscope from being widely adopted.

Moreover, since the antennas are attached to the patient's abdomen, there is problem with the sensitivity of the antennas in receiving signals. For instance, the capsular endoscope tends to have lowered sensitivity in wireless data transmission when it is located in an area of one meter into the small intestine from the pylorus.

Technically speaking, the wireless data transmission employed in human body must be done at low frequency to avoid any adverse influence on the human body. Therefore, it is impossible to allow a quite high data transmission volume and a large width for each unit of data for the conventional capsular endoscope. As a result, the number of pictures that can be taken per second, and the picture resolution are largely restricted. In some areas, such as the esophagus, the capsular endoscope moves faster, and some messages might be missed since the endoscope always takes only two pictures within one second. In addition, it also impossible to install too many pieces of image-sensor chips in the capsular endoscope for capturing more image data in real time for the doctor to have sufficient information to make a diagnostic determination.

It is therefore desirable to develop a capsulated endoscope with memory storage device to overcome the drawbacks existed in the conventional capsular endoscope. In the capsulated endoscope with memory storage device, there is provided storage space for storing the pictures taken in real time, so that no radio transmitter and external antennas and wireless receiver are needed to largely reduce the cost and increase the convenience in use of the endoscope.

### SUMMARY OF THE INVENTION

A primary object of the present invention is to provide a capsulated endoscope with memory storage device, with which image pictures taken by the endoscope is stored in a memory module without the need of being transmitted to external antennas and wireless receiver via any internal radio transmitter to largely reduce the cost and increase the convenience of the capsulated endoscope.

Another object of the present invention is to provide a capsulated endoscope with memory storage device, an internal controller of which is connected to the memory storage device in a data bus interface mode, so as to control and access the memory storage device. In this manner, data stored in the memory storage device can be accessed at an extremely high speed and in a very large data volume. Therefore the number of pictures that can be taken per second and the image resolution of the pictures taken by the capsulated endoscope with memory storage device is increased.

A further object of the present invention is to provide a capsulated endoscope with memory storage device, in which more than one image-sensor chip is provided near front and rear ends of the capsulated endoscope to cooperatively take pictures in almost 360 degrees to provide complete image data for a doctor to make accurate determination from the image data.

To achieve the above and other objects, the capsulated endoscope with memory storage device according to the present invention includes a capsular main body of 11×26 mm in size, which is internally provided with a computer host interface, a controller connected to the computer host interface, a image-sensor chip connected to the controller, a plurality of LEDs, a memory module, and a battery for supplying power. The capsular main body is acid and alkali resisting, and includes a front optically transparent dome to serve as a viewing window. When the capsular main body is swallowed for conducting an examination, it moves forward with the peristalsis of intestine. While the capsulated endoscope moves forward, it flashes more than twice per second for the image-sensor chip to take pictures inside the examined organs at the same time. The image signals obtained by the image-sensor chip are then written into the memory module at an extremely high speed.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a functional-block diagram showing the structure of a capsulated endoscope with memory storage device according to the present invention;

FIG. 2 is perspective view of the capsulated endoscope with memory storage device according to the present invention; and

FIG. 3 is a cutaway view of the capsulated endoscope with memory storage device according to the present invention.

### DETAILED DESCRIPTION OF THE PREFERRED EMBODIMENTS

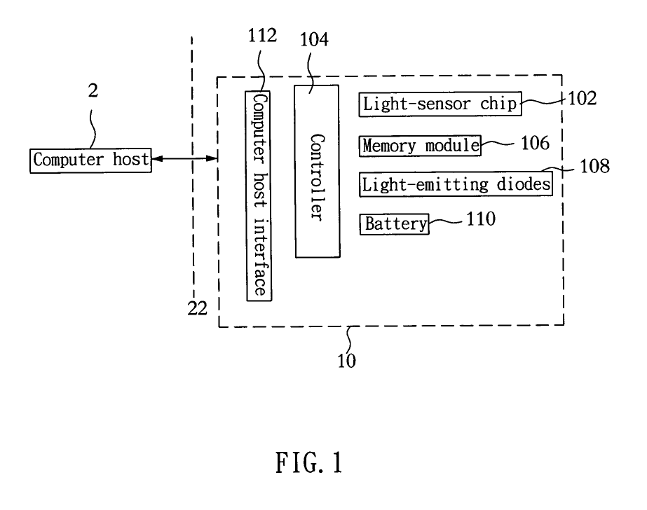

Please refer to FIG. 1 that is a functional-block diagram of a capsulated endoscope with memory storage device according to an embodiment of the present invention. As shown, the capsulated endoscope of the present invention includes a capsular main body 10, which is internally provided with a image-sensor chip 102 for capturing images and taking pictures, a controller 104 for compressing the pictures taken by the image-sensor chip 102, a memory module 106 for storing the compressed pictures transmitted thereto by the controller 104, a plurality of light-emitting diodes (LEDs) 108 located near a front end of the capsular main body 10 to provide sufficient light source for the image-sensor chip 102 to capture images and take pictures, a battery 110 for supplying power to all the components in the capsular main body 10, and a computer host interface 112 for electrically connecting to a universal serial bus (USB) 22 on an external computer host 2 to enable transmission of image data stored in the memory module 106 to the computer host 2 for display on a monitor.

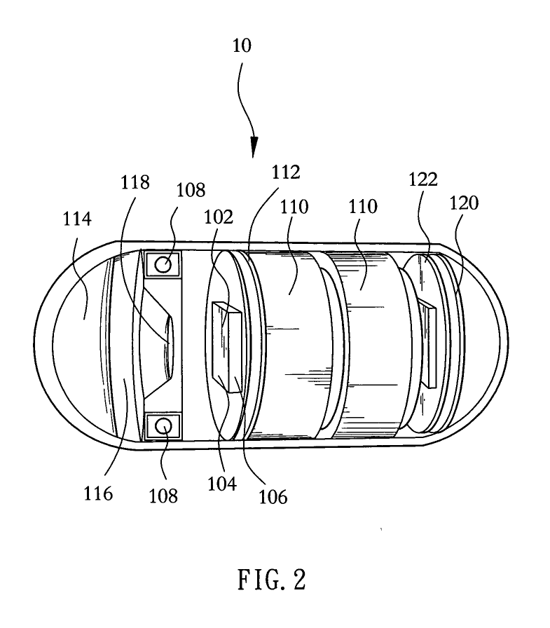

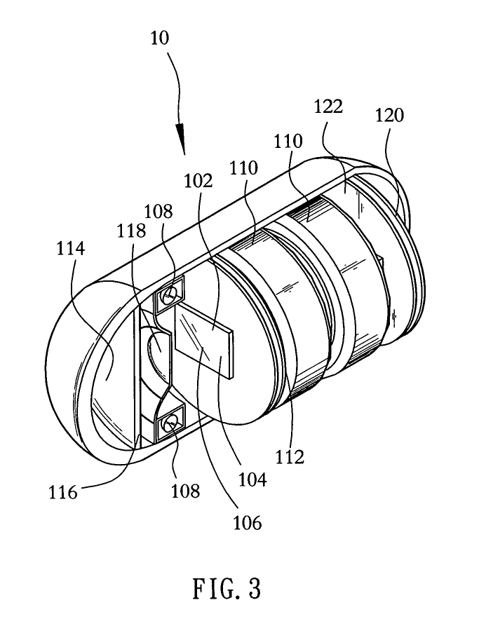

Please refer to FIGS. 2 and 3 that are perspective and cutaway views, respectively, of the capsulated endoscope with memory storage device according to the present invention. As shown, the capsular main body 10 has an optically transparent dome 114 located at the front end thereof. A lens holder 116 is located behind the dome 114 for holding a lens 118 thereon. The LEDs 108 are spaced around a rear side of the lens holder 116. When the capsular main body 10 is swallowed, light emitted from the light-emitting diodes 108 enables the taking of pictures of clear images. The image-sensor chip 102 is located behind the lens 118. In the present invention, the image-sensor chip 102, the controller 104, and the memory module 106 are integrated into a single chip through semiconductor manufacturing techniques to largely reduce an overall volume thereof, enabling the storage of large amount of image data at low power consumption without causing any adverse influence on human body by high frequency. The computer host interface 112 is located behind the integrated controller 104, image-sensor chip 102, and memory module 106 to serve as a bridge between the controller 104 and the computer host 2. An additional memory module 120 and an expansion slot 122 may be further provided in the rearmost space in the capsular main body 10 to expand the memory capacity of the capsulated endoscope.

In the present invention, the computer host interface 112 is a USB interface corresponding to the USB 22. However, the computer host interface 112 may be an IEEE 1394 interface or other standard interfaces, too. The computer host interface 112 is connectable to the USB 22 on the computer host 2, and the controller 104 is connected to the computer host interface 112. Thus, the controller 104 may be connected to the computer host 2 via the computer host interface 112. The controller 104 is also connected to the memory module 106. In the illustrated embodiment, the memory module 106 is an NAND Flash interface. However, the memory module 106 may be a Smart Media interface, a Memory Stick interface, an SD interface, an xD interface, or other standard storage device interface. Moreover, in the illustrated embodiment, the image-sensor chip 102 maybe a Complementary Metal-Oxide Semiconductor (CMOS) image sensor, a Charge-Coupled Device (CCD) image sensor, or other types of imaging devices.

The controller 104 serves to communicate with the computer host 2, and manage the memory module 106, the LEDs 108, and the image-sensor chip 102. The memory module 106 must include at least one storage chip or memory for storing data, such as a flash memory, a programmable read-only memory (PROM), or any electrically erasable programmable read-only memory (EEPROM). When the battery 110 in the capsular main body 10 is actuated to supply power, the controller 104 drives the LEDs 108 and the image-sensor chip 102 to continuously take pictures and immediately store the captured images on the memory module 106 in real time. The controller 104 also manage the memory module 106 at the same time, so that the image pictures taken by the image-sensor chip 102 are stored in different image files, each of which contains a predetermined number of pictures taken. With these arrangements, the capsulated endoscope is able to faithfully record all pictures it takes in the patient's body, from the esophagus to the small intestine or even the large intestine, until the battery is exhausted. The capsulated endoscope is eventually discharged from the anus along with excrements to complete the whole examination.

The capsulated endoscope discharged from the anus is washed and disinfected. Then, the capsular main body 10 is cut open, and the computer host interface 112 is connected to the computer host 2. The controller 104 in the capsulated endoscope communicates with the computer host 2 via the computer host interface 112. When the controller 104 is connected to the computer host 2, it also obtains power supply from the computer host 2 to read an information block into a static random-access memory (SRAM) of the controller 104. According to the data in the information block loaded into the SRAM, the controller 104 responds to the request made by the computer host 2 to allocate the memory module 106 and treat the memory module 106 as a logic disk. The computer host 2 freely accesses all image files via the logic disk. Suitable applications in the computer host 2 would operate in multiplexing to continuously retrieve all image files on the memory module 106 and display the retrieved image files on a monitor in real time. The displayed image files are then processed before being stored on a compact disk.

In the present invention, the controller 104 functions to control both the image-sensor chip 102 and the memory module 106, and the image data on the image-sensor chip 102 controlled by the controller 104 are read via a data bus interface, and the read image data are written into the memory module 106, which is on the same data bus interface, at an extremely high speed to enable largely increased number of frames of pictures taken per second. In the present invention, there may be more than one image-sensor chip 102 separately internally provided near front and rear ends of the capsular main body 10 to cooperatively take image pictures in almost 360 degrees. In this manner, more complete image information can be obtained for a doctor to make accurate determination from the images.

With the memory module 106 provided in the capsular main body 10, the capsulated endoscope of the present invention is able to take and store pictures in real time without the need of any radio transmitter and antenna for receiver. Thus, the examination using the capsulated endoscope can be highly conveniently conducted at largely reduced cost, and the patient undergoing the examination need not carry any examination instrument and is allowed to move freely. Moreover, in the present invention, the controller 104, the image-sensor chip 102, and the memory module 106 are integrated into a single chip through the currently available semiconductor manufacturing techniques to largely reduce the overall volume thereof, which in turn enables the capsulated endoscope to take and store image pictures in large data volume, and the number of pictures that can be taken per second can be largely increased, or a image-sensor chip 102 with high resolution can be employed. In the present invention, the battery 110 may includes a plurality of mercury cells, or rechargeable lithium cells or lithium polymer cells. Further, the capsular main body 10 is an acid and alkali resisting shell made through nano science and technology.

The present invention has been described with a preferred embodiment thereof and it is understood that many changes and modifications in the described embodiment can be carried out without departing from the scope and the spirit of the invention that is intended to be limited only by the appended claims.

What is claimed is:

1. A capsulated endoscope with memory storage device, comprising:
a capsular main body being internally provided with at least one image-sensor chip 102 connected to a controller for capturing images in organs of a patient undergoing an examination, a plurality of light-emitting diodes (LEDs) located near a front end of said capsular main body for providing a light source for said image-sensor chip to capture images and take pictures, a computer host interface located behind said controller 104 for connecting to and communicating said controller with an external computer host, and a memory module; and
said memory module being connected to said controller, so that data of images captured by said at least one image-sensor chip is transmitted to and stored on said memory module via said controller.

2. The capsulated endoscope with memory storage device as claimed in claim 1, wherein said controller, said memory module, and said image-sensor chip are integrated into a single chip through semiconductor manufacturing techniques.

3. The capsulated endoscope with memory storage device as claimed in claim 1, wherein said image-sensor chip is more than one in number for separately mounting near a front and a rear end of said capsular main body to cooperatively take pictures in almost 360 degrees and thereby provide more complete image information about the organs being examined.

4. The capsulated endoscope with memory storage device as claimed in claim 1, wherein said computer host interface is selected from the group consisting of universal serial bus (USB) interface, IEEE 1394 interface, and other standard interfaces.

5. The capsulated endoscope with memory storage device as claimed in claim 1, wherein said memory module is selected from the group consisting of NAND Flash interface, Smart Media interface, Memory Stick interface, SD interface, xD interface, and other standard storage device interfaces.

6. The capsulated endoscope with memory storage device as claimed in claim 1, wherein said image-sensor chip is selected from the group consisting of CMOS image sensor, CCD image sensor, and other imaging devices.

7. The capsulated endoscope with memory storage device as claimed in claim 1, further comprising an expansion slot provided near the rear end of said capsular main body.

8. The capsulated endoscope with memory storage device as claimed in claim 7, further comprising an additional memory module adapted to insert into said expansion slot to provide an expanded a memory capacity.

9. The capsulated endoscope with memory storage device as claimed in claim 1, wherein said capsular main body is an acid and alkali resisting shell made through nano science and technology.

10. The capsulated endoscope with memory storage device as claimed in claim 1, further comprising a battery, and said battery is selected from the group consisting of mercury cells, rechargeable lithium cells, and rechargeable lithium polymer cells.

11. The capsulated endoscope with memory storage device as claimed in claim 1, further comprising an optically transparent dome located at the front end of said capsular main body, a lens holder mounted in said capsular main body behind said transparent dome, and a lens mounted on said lens holder, allowing said image-sensor chip to capture images in widened areas.
